(19) United States
(12) Patent Application Publication
(10) Pub. No.: US 2008/0261298 A1
(43) Pub. Date: Oct. 23, 2008

(54) Method and device for the multiplex analysis of cells and tissues

(75) Inventor: Hiroyuki Yonekawa, Center Valley, PA (US)

Correspondence Address: Hiroyuki Yonekawa, 3500 Corporate Pkw., PO Box 610, Center Valley, PA, 18034, US

(73) Assignee: Olympus American Inc., Center Valley, PA (US)

(21) Appl. No.: 11/788,829

(22) Filed: Apr. 23, 2007

(51) Int. Cl. C12M 3/00

(52) U.S. Cl. 435/288.4

(57) ABSTRACT

This invention is used for the multiplex analysis method of cells or tissues by combination of multiple cubicles and permeable membrane. Utilizing the diffusion based reagents administration; cells and tissues in multiple cubicles receive signals evenly in their cubicles as parallel manner. This device makes efficient multiplex cells or tissues assays with simple plate layout. According to the molecular permeable mechanism, single spike of stimuli as chemical trigger, light activation, electric triggers or temperature shift can induce multiple cells or tissues response at once. Then accompanying with calibrator molecules as for the navigation of stimuli and assisted by suitable computer simulation, accurate kinetic cells or tissues response analysis is achieved.

### BACKGROUND OF THE INVENTION

The cell culture technique has been used in many biological research fields and it expands their utilities. Different cells, tissues, culture medium and culture wears are use in order to increase the efficiency of output. According to the necessity of assays volume, multiple well plates were incorporated in past 30 years.

Then, according to the achievement of more native cell or tissue environment, 3D cell culture technique is also getting popular in cells or tissue cultures. Special culture wares were invented for the efficient 3D cell culturing technique too. (Ref 1-5)

By the complexity of 3D culture, the multiple processing and image acquisition of cells or tissues are quite difficult. In this patent application, we apply the permeable membrane with separate cubicles for cell culture, especially 3D cell culture for increasing output by incorporating multiplexing assays. Then this patent application shows a successful approach to establish the efficient multiplex assay method for cells or tissue analysis.

### SUMMARY OF THE INVENTION

At first, multiple cubicles are formed in the bottom of microplate or culture insert. Then permeable membranes are installed in order to separate these cubicles. Such permeable membranes can form the extra-space for the reagent additional part between these cubicles too. This extra-space is used for the chemical administration and drug-releasing portion. Cells or tissues exist in each cubicle and they are observed by conventional optical modality as well as more 3D adaptable optics. Multi-photon observation is preferable. Additional calibration molecules as colored dyes or fluorescence molecules assist more accurate observation. Then, computer assisted simulation will compensated the data and increase the quality of results.

### EMBODIMENT 1

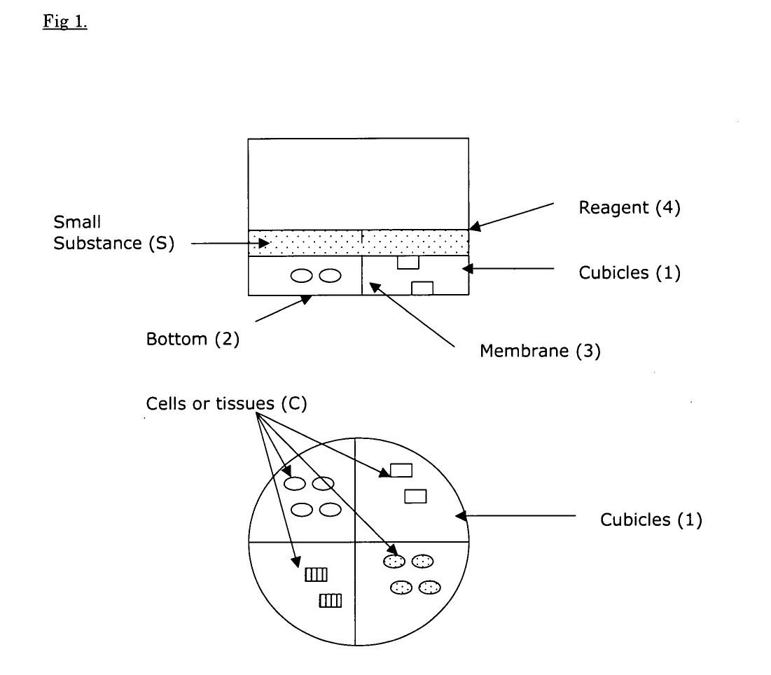

### Multiplex Analysis of Cells or Tissue in Single Reaction Vessel (FIG. 1).

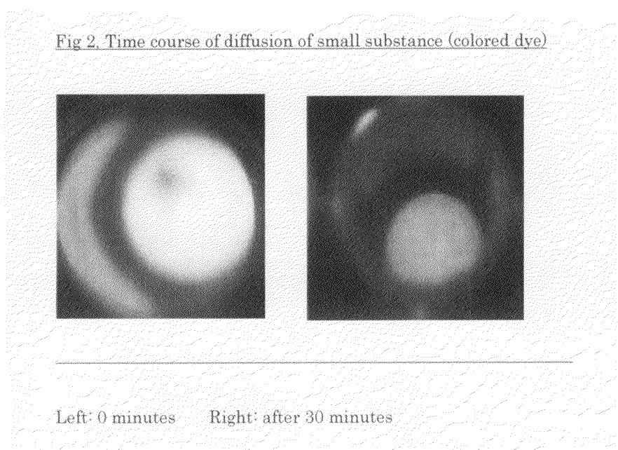

Multiple cubicles (1) are formed in the bottom of culture well or culture insert (2). The permeable membrane (3) separates each cubicle (1). Cells or tissues (C) are located in each cubicles (1) relatively stable manner. If the culture medium contains gelling materials, these cells or tissues are more stable in each cubicle (1). The bottom (2) of the cubicles is transparent for the optical measurement from bottom side. After the administration of reagent (4) on the top layer of cubicles (1), small molecules (S) permeate into the cubicles by time dependent manner. And all cells or tissues (C) receive stimulus uniformly. According to the simple experiments, the diffusion time needs about 30 minutes crossing the 8 mm traveling when the medium contains 0.5% gelatin in PBS/0.1% Tween 20 at room temperature (FIG. 2). The kinetics of diffusion speed varies by their environment as temperature, ionic concentration, culturing materials and molecular weight of substances. So the measurement is compensated by calibrator dyes and computer simulation.

### EMBODIMENT 2

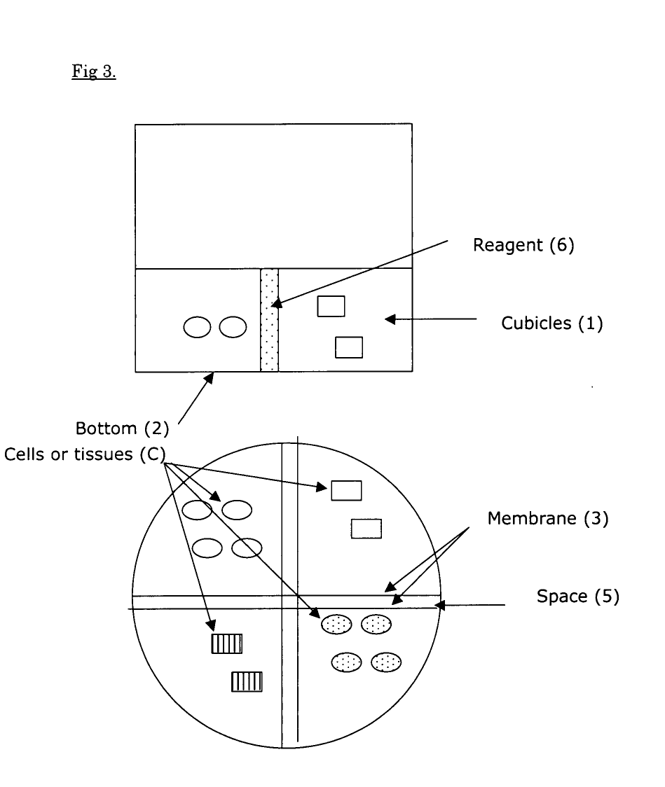

### Multiple Analysis of Cells or Tissue with Extra Reagent Space in Single Reaction Vessel (FIG. 3)

Multiple cubicles (1) are formed in the bottom of culture well or culture insert (2). The permeable membrane (3) separates each cubicle (1) and prepares extra space (5). Cells or tissues (C) are located in each cubicle (1). The bottom (2) of the cubicles is transparent for the optical measurement from. Reagent (6) is administrated into the extra space (5). Small molecules (S) permeate into the all cubicles by time dependent manner and cells or tissues (C) receive stimuli evenly. The kinetics of diffusion speed varies by their environment as temperature, ionic concentration, culturing materials and molecular weight of substances. So the measurement is compensated by calibrator dyes and computer simulation.

### CROSS REFERENCE

this patent application is a subsequent application to the U.S. patent application Ser. No. 11/698,966. Filed Jan. 29, 2007.
Ref 1: U.S. Pat. No. 5,583,037
Ref 2: U.S. Pat. No. 5,665,596
Ref 3: U.S. Pat. No. 5,580,781
Ref 4: U.S. Pat. No. 6,939,709
Ref 5: U.S. Pat. No. 6,998,265

1. Method of multiplex cells or tissue analysis with the culture vessel of separate compartment cubicles and permeable materials.

2. The method of multiplex cells or tissue analysis of claim 1, wherein said method is Kinetic analysis method by evoked with diffusing chemicals.

3. The method of multiplex cells or tissue analysis of claim 1, wherein said outside signals contain diffusible small molecules, light activation, electrical activation, temperature shift or mechanical manipulations.

4. The method of multiplex cells or tissue analysis of claim 1, wherein said permeable materials are membrane filter, ceramic filter, glass filter or biological polymers.

5. The method of multiplex cells or tissue analysis of claim 1, wherein said compartment cubicles contains 3D matrices as gelatin, fibrin or Matrigel.

7. The method of multiplex cells or tissue analysis of claim 1, wherein said method is accompany with the navigation molecules as colored dyes or fluorescent dyes as kinetic calibrator.

8. The method of multiplex cells or tissue analysis of claim 1, wherein said the data of kinetic analysis is compensated by computer simulation technique.
